(19) United States
(12) Patent Application Publication
(10) Pub. No.: US 2024/0148551 A1
(43) Pub. Date: May 9, 2024

(54) Infusion Sleeve For Phacoemulsification

(71) Applicant: Bausch & Lomb Ireland Limtied, Dublim (IE)

(72) Inventor: Toh Seng GOH, Wildwood, MO (US)

(21) Appl. No.: 18/377,451

(22) Filed: Oct. 6, 2023

Related U.S. Application Data
(60) Provisional application No. 63/414,124, filed on Oct. 7, 2022.

(51) Int. Cl. A61F 9/007

(52) U.S. Cl. CPC A61F 9/00745; A61B 2217/007

(57) ABSTRACT

An ophthalmic surgical handpiece includes a needle for insertion through an incision in an eye and a generator of ultrasonic energy for supplying the ultrasonic energy to the needle to emulsify tissue within the eye. The ophthalmic surgical handpiece also includes a sleeve surrounding at least a portion of the needle, the sleeve including an insertion portion for supplying infusion fluid into the eye and a compressible portion for sealing the incision in the eye by pushing against an exterior surface of the eye at the incision.

### FIELD

The present disclosure generally relates to infusion sleeves for phacoemulsification, and more particularly, to infusion sleeves for phacoemulsification that enables a phacoemulsification needle to move independently of the infusion sleeve.

### BACKGROUND

This section provides background information related to the present disclosure which is not necessarily prior art.

Ophthalmic surgery often involves cutting away or emulsifying tissues that need to be removed from the eye, such as in cataract surgery. One known technique is phacoemulsification (e.g., coaxial phacoemulsification, etc.). This technique involves using high frequency ultrasound energy transmitted through a handpiece into a phaco needle to emulsify affected tissue within the eye, and the emulsified tissue is then aspirated out of the eye through the phaco needle. To maintain the eye in a pressurized condition during surgery and to aid in the aspiration of the emulsified tissue, an infusion fluid (e.g., a saline fluid) is introduced into the eye through the handpiece. In particular, the fluid flows into the eye through an infusion sleeve that is positioned around the phaco needle (e.g., coaxial with the phaco needle). The infusion sleeve and the phaco needle are both inserted into the eye through an incision in the eye.

### SUMMARY

This section provides a general summary of the disclosure, and is not a comprehensive disclosure of its full scope or all of its features.

Example embodiments of the present disclosure generally relate to infusion sleeves for ophthalmic surgical instruments (e.g., phacoemulsification handpieces, etc.). In one example embodiment, an ophthalmic surgical instrument includes a needle for insertion through an incision in an eye and a generator of ultrasonic energy for supplying the ultrasonic energy to the needle to emulsify tissue within the eye. The ophthalmic surgical handpiece also includes a sleeve surrounding at least a portion of the needle, the sleeve including a distal insertion portion for supplying infusion fluid into the eye and a more proximal compressible portion for sealing the incision in the eye by pushing against an exterior surface of the eye at the incision.

In another example embodiment, an infusion sleeve generally includes an insertion portion for insertion into an incision of an eye and a more proximal compressible portion for sealing against an exterior surface of the eye at the incision. The insertion portion has a first diameter and the compressible portion has a second diameter that is larger than the first diameter.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a partial perspective view of an example embodiment of an infusion sleeve partially surrounding a phacoemulsification needle;

FIG. 2 is a cross-section elevation view of another example embodiment of an infusion sleeve partially surrounding a phacoemulsification needle compared to a prior art example view, with the tips of the infusion sleeves and the phaco needles inserted into an eye to a first depth;

FIG. 3 is a cross-section elevation view of the infusion sleeves of FIG. 2, with the tips of the needles inserted into the eye to a second depth;

FIG. 4 is a cross-section elevation view of the infusion sleeves of FIG. 2, with the tips of the phaco needles fully inserted into the eye to a third depth.

Corresponding reference numerals indicate corresponding parts throughout the several views of the drawings.

### DETAILED DESCRIPTION

Example embodiments of the present disclosure generally relate to infusion sleeves for phacoemulsification handpieces that provide infusion fluid into an eye from a consistent location relative to an incision site as well as structure for sealing the incision site to prevent or minimize leakage of fluid from the incision, regardless of the size of the incision site. Conventionally, an infusion sleeve is designed to have an interference fit with the phaco needle and move in parallel along with the phaco needle during surgery (e.g., the phaco needle and infusion sleeve both move forward and backward together, etc.). As a result, the infusion fluid enters the eye at a location near the tip of the phaco needle, and consequently near the location where emulsified tissue is aspirated from the eye by the phaco needle. However, the introduction of fluids from the infusion sleeve near the tip of the phaco needle may be counterproductive, for example, as the infusion fluid may push fragments of tissue that must be emulsified and aspirated away from the tip of the phaco needle. Additionally, a drop in intraocular pressure (IOP) when infusion fluid leaks out of the incision, may cause considerable problems such as collapse of the intraocular tissue onto the vibrating phaco needle. To prevent such a leak, infusion sleeves have historically been designed to mostly correspond to the size of the incision (e.g., to seal the incision by eliminating the space between the sleeve and the incision site, etc.). This necessitates differently sized infusion sleeves for a range of incision sizes. Moreover, in a typical phacoemulsification procedure, the phaco needle and infusion sleeve move forward and backward along the incision an average of fifty times. When the phaco needle is moving forward and backward, the sleeve rubs at the internal incision surface, which may induce tissue damage (e.g., corneal burns) and negatively impact post-surgery healing. Further, the visibility of needle and surgical field may also be compromised with the sleeve moving along with the phaco needle during surgery.

Uniquely, the infusion sleeves of the present disclosure include an insertion portion that is inserted through the incision and inside of the eye, as well as a collapsible or compressible portion that remains outside of the eye and deforms (e.g., collapses or accordions) when the phaco needle advances further into the eye. Due to this dual-segmented construction, the infusion sleeve of the present disclosure enables the phaco needle to move forwards and backwards into the eye as desired during surgery, while the infusion sleeves remains stationary, for example, with only the insertion portion of the infusion sleeve positioned within the eye. Because the infusion sleeve remains stationary even when the phaco needle is moving, damage to ocular tissues at the incision site is minimized or eliminated (e.g., the infusion sleeve does not continuously rub against the ocular tissues at the incision site during surgery, etc.). Additionally, the external compressible portion of the infusion sleeve functions to seal the incision from the outside. As can be appreciated, this external sealing enables a single combination of an infusion sleeve and phaco needle to be used with wide range of incision sizes.

Example embodiments will now be described more fully with reference to the accompanying drawings. The description and specific examples included herein are intended for purposes of illustration only and are not intended to limit the scope of the present disclosure.

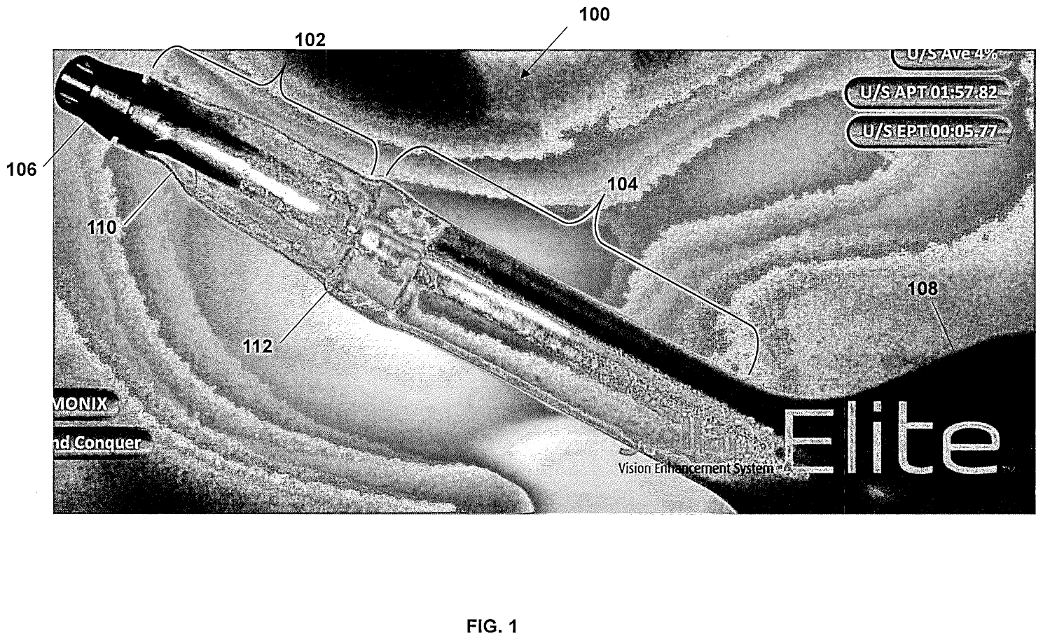

FIG. 1 illustrates an example embodiment of an infusion sleeve 100 for ophthalmic surgical instruments (e.g., phacoemulsification handpieces, etc.) including one or more aspects of the present disclosure that is a hand-made prototype sleeve. In the illustrated embodiment, the sleeve 100 generally includes an insertion portion 102 (broadly, a first or internal portion) and a compressible portion 104 (broadly, a second or external portion). A needle 106 (e.g., a phaco needle, etc.) of an ophthalmic surgical instrument 108 (e.g., a phacoemulsification handpiece, etc.) is positioned within the sleeve 100. In connection with an ophthalmic surgical procedure, the insertion portion 102 of the sleeve 100 is inserted into an incision (broadly, an opening) in the eye along with the needle 106, while the compressible portion 104 of the sleeve 100 remains outside of the eye.

The sleeve 100 is generally formed as a hollow tube. In particular, the sleeve 100 includes a central opening to accommodate the needle 106 within the sleeve 100. During a surgical procedure, infusion fluid passes through the sleeve 100 and is supplied to the eye to maintain intraocular pressure. The infusion fluid enters the eye through one or more infusion ports 110 of the sleeve 100. In particular, in the illustrated embodiment, the insertion portion 102 of the sleeve 100 includes at least one infusion port 110 through which infusion fluid is introduced into the eye. The infusion port 110 is formed proximal of the distal end of the sleeve 100. In the illustrated embodiment, the distal end of the insertion portion 102 of the sleeve 100 is tapered, for example, to facilitate easier insertion of the sleeve 100 through the incision and into the eye.

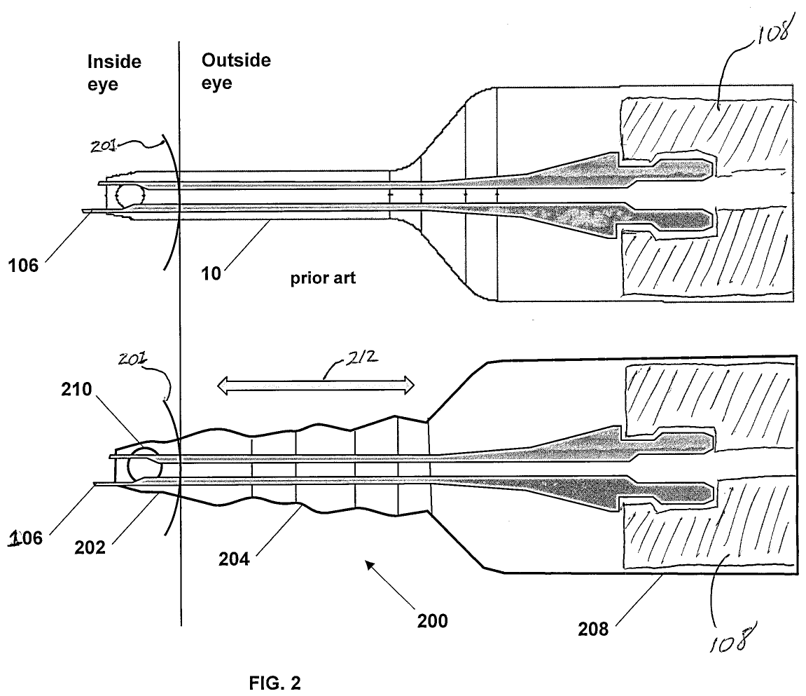

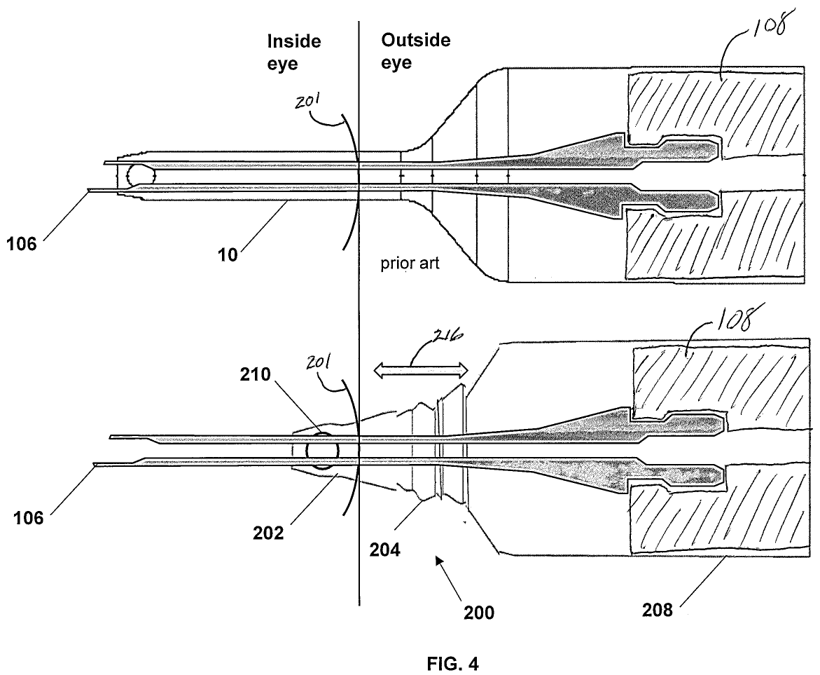

In the illustrated embodiment, the insertion portion 102 is of a smaller diameter than the compressible portion 104 of the sleeve 100. The diameter of the insertion portion 102 must be small enough to fit within the incision without stretching or tearing the incision, while also being large enough to receive the phaco needle 106 within the sleeve 100. In some embodiments, due to the difference in diameter, a lip 112 is present at the distal end of the compressible portion 104. In other embodiments, the transition between the different diameters of the insertion portion 102 and the compressible portion 104 is smooth, such that there is not a lip or step between the two portions 102, 104 but rather more of a flare from the smaller first diameter to the larger second diameter. The second diameter of the sleeve's compressible portion 104 is sized large enough not to pass through the incision and the compressible portion is configured to remain external to the eye during surgery. The insertion portion 102 of the sleeve 100 is able to enter the eye through an incision due to its relatively smaller diameter, while the compressible portion 104 of the sleeve 100 remains outside of the eye due to its relatively larger diameter (e.g., the lip 112 engages with the exterior surface of the eye to prevent the compressible portion 104 from entering the eye, etc.). When the phaco needle is fully inserted into the eye (e.g., FIG. 4), the compressible portion 104 of the sleeve 100 is positioned outside of the eye and is compressed (e.g., due to uniaxial compression, etc.), such that the effective length of the compressible portion 104 decreases. In some embodiments, the compressible portion 104 of the sleeve 100 includes fold lines or other structures to guide the shape of the compressible portion 104 as the sleeve 100 is compressed (e.g., such that the compressible portion 104 deforms in an accordion-like manner, etc.), as in the example embodiment of FIGS. 2-4.

The compressible portion 104 of the sleeve 100 functions as a compressible seal for the incision, where the incision is sealed by the portion of the sleeve 100 that is outside of the eye (i.e., the compressible portion 104). In particular, rather than functioning as a plug to seal the incision (e.g., sealing by pushing against the inside of the incision, etc.), the sleeve 100 functions as a cap to seal the incision (e.g., sealing by pushing against the outside of the incision, etc.). In particular, the compressible portion 104 of the sleeve 100 compresses against the exterior surface of the eye and the compressible portion 104, when compressed, pushes against the exterior surface of the eye at and around the incision, effectively sealing the incision. To achieve the seal, the diameter of the compressible portion 104, is larger than the incision, such that the compressible portion 104, when collapsed entirely covers the incision from the outside of the eye (e.g., rather than filling up the incision from within, etc.). In some embodiments, the lip 112 of the compressible portion 104 is of a sufficiently large diameter to seal the incision as a cap, for example, independent of the diameter of the compressible portion 104 when compressed. In these embodiments, the lip 112 may be considered a flange at the distal end of the compressible portion 104 that is of a diameter large enough to surround the incision (and, additionally or alternatively, of a diameter greater than the rest of the compression portion 104 when compressed). For example, the distal surface of such flange seals against the exterior surface of the eye to seal the incision. As can be appreciated, a single combination of an infusion sleeve 100 and phaco needle 106 can be used with wide range of incision sizes, for example, due to the different diameters of the insertion portion 102 and the compressible portion 104 as well as the sealing mechanism of the sleeve 100 (e.g., by externally sealing the incision, etc.). Moreover, fluidics stability may be improved due to sealing the incision at the external eye surface with the compressible portion 104 of the sleeve 100 and the larger diameter of the compressible portion 104 may provide enhanced infusion into the eye compared to a typical prior art sleeve of mostly constant diameter.

The compressible portion 104 of the sleeve 100 is preferably formed of a compressible bio-compatible material that externally seals against the incision when the insertion portion 102 of the sleeve 100 (and the phaco needle) is inserted through the incision. The compressible section 104 of the sleeve 100 is intended to be easily deformed or compressed and, as such, the compressible section 104 may be described as flexible, compressible, elastic, pliable, and/or deformable. For example, the compressible section 104 may be formed of a compressible material, including without limitation, silicone, rubber, plastic, polymers, nylons, polyethers, polyurethanes, etc. If the sleeve 100 is formed of two parts joined together, the insertion portion 102 may be formed of stiffer, more rigid material to enable easier insertion through the incision. It should be appreciated that the compressible section 104 may be formed of any material and/or combination of materials that are effective for phaco surgery.

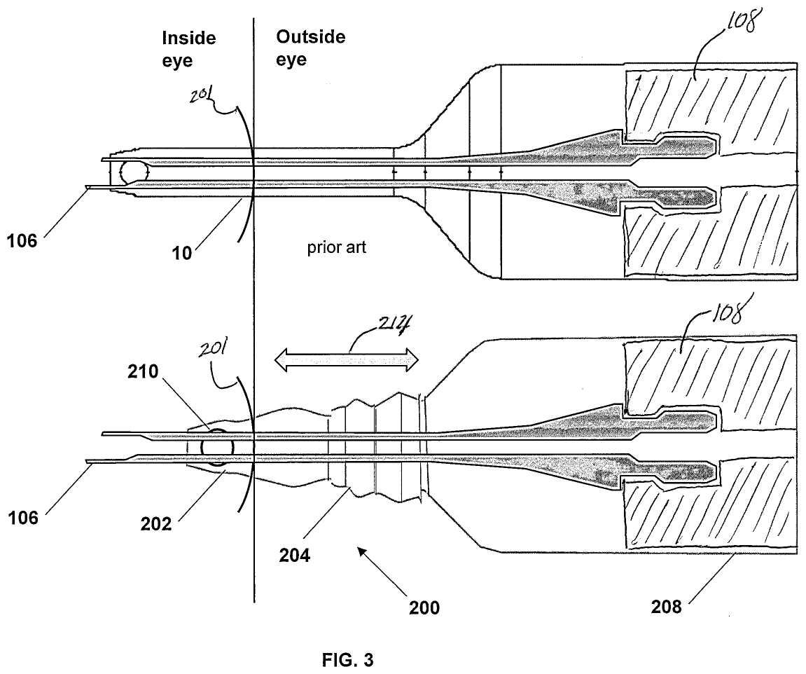

FIGS. 2-4 illustrate insertion of a phaco needle 106 attached to a phaco handpiece 108 and an infusion sleeve 200 into an eye through an incision. In particular, FIG. 2 illustrates the phaco needle 106 as inserted into an eye 201 to an initial or first depth. FIG. 3 illustrates the phaco needle 106 as inserted to a second deeper depth, and FIG. 4 illustrates the phaco needle 106 as fully inserted to a third deepest depth. For purposes of comparison, an identical phaco needle 106 that includes a conventional infusion sleeve 10 is labeled as prior art and shown at each corresponding depth and is depicted above the infusion sleeve 200 of the present disclosure in FIGS. 2-4.

As shown in FIG. 2, when the phaco needle 106 is initially inserted into the eye, the distal insertion portion 202 of the sleeve 200 is positioned within the eye and the compressible portion 204 is positioned outside of the eye. Because at least a second diameter of the compressible portion 204 is larger than at least a first diameter of the insertion portion 202 (and larger than the incision in eye 201), the compressible portion 204 never enters the eye and prevents more of the sleeve 200 from entering the eye 201. The diameter of the sleeve's compressible portion is sized large enough not to pass through the incision and the compressible portion is also configured to remain external to the eye during surgery. As shown in FIG. 3, when the phaco needle 106 advances into the eye (e.g., to a second depth, etc.), the sleeve 200 does not advance further into the eye. In particular, the insertion portion 202 of the sleeve 200 remains in the same position within the incision. The first diameter of the sleeve's insertion portion is sized to be inserted through the incision and does not seal the incision of the eye. To enable the phaco needle 106 to advance without the sleeve 200 advancing, the compressible portion 204 of the sleeve 200 decreases in effective length, as shown by arrow bars 212, 214, 216. In contrast, as shown in FIG. 3, when the phaco needle 106 advances into the eye to the second depth, the conventional sleeve 10 also advances into the eye. This creates friction at the incision as the conventional sleeve 10 rubs against the incision moving back and forth between the first depth to the second depth, etc.

Similarly, in FIG. 4, the phaco needles 106 are fully inserted to a third depth. Again, the insertion portion 202 of the sleeve 200 remains in the same position with respect to the exterior surface of the eye (e.g., the same position as shown in FIGS. 2 and 3, etc.). In this way, the phaco needle 106 is able to be inserted to a greater depth, independent of the sleeve 200, which may improve the ability to emulsify the lens material as desired (e.g., improved cracking and chopping, better holding power (purchase), etc.). In contrast, when the phaco needle 106 is fully inserted, the conventional sleeve 10 is likewise fully inserted. Comparing the sleeve 200 to the conventional sleeve 10, a relatively smaller length of the sleeve 200 enters the eye 201 (e.g., only the insertion portion 202 of the sleeve 200, etc.). Because a shorter length is inserted, the insertion portion may be more likely to be easily inserted through the incision without buckling because of the effective stronger structural strength compared to conventional sleeve 10 that has a uniform cross-section of much greater length. Moreover, the stationary positioning of the infusion sleeve 200 during surgery (e.g., independent of the movement of the phaco needle 106, etc.) enables infusion fluid to be introduced into the eye 201 in a consistent location relative to the incision site through the infusion port 210. In particular, infusion is further away from the tip of the phaco needle 106, as compared to the conventional infusion sleeve 10 at all but the initial insertion depths. This may enhance the emulsification and aspiration of tissues from the eye, because the tissue is not pushed or directed away from the tip the phaco needle 106 by fluid exiting port 210, as may happen with conventional sleeve 10.

Infusion sleeves of the present disclosure include a unique dual-portion structure that includes an insertion portion that is inserted in the eye through the incision and an external portion that remains outside of the eye as the phaco needle advances into the eye and compresses to seal against the incision from outside of the eye. Due to this dual-portion construction, the infusion sleeve of the present disclosure enables the phaco needle to move forwards and backwards into the eye as desired during surgery, while the infusion sleeve remains stationary, for example, with only the insertion portion of the infusion sleeve positioned within the eye. Because the infusion sleeve remains stationary even when the phaco needle is moving, damage to ocular tissues at the incision site is minimized or eliminated (e.g., the infusion sleeve does not continuously rub against the ocular tissues at the incision site during surgery, reducing the risk of a corneal burn).

Moreover, the stationary positioning of the infusion sleeve of the present disclosure during surgery (e.g., independent of the movement of the phaco needle, etc.) enables infusion fluid to be introduced into the eye in a consistent location relative to the incision site. In particular, infusion is further away from the tip of the phaco needle, as compared to a conventional infusion sleeve during most of a surgical procedure. This ensures that emulsified or fragmented tissues are efficiently aspirated from the eye, rather than pushed or directed away the needle tip by fluid exiting the aspiration port. Additionally, because the phaco needle is able to be inserted to a depth independent of the depth to which the infusion sleeve is inserted, the infusion sleeve of the present disclosure enables the phaco needle to be more deeply embedded into the lens material with improved views of the needle tip and tissue (because the sleeve is not in the visual field of interest), thus improving the ability to identify and emulsify the lens material as desired (e.g., improved cracking and chopping, better holding power, etc.). The independence of the phaco needle from the infusion sleeve also improves the visibility of the phaco needle (and broadly, the surgical field) during surgery.

As can be appreciated, a single combination of a phaco needle and an infusion sleeve of the present disclosure can be used with wide range of incision sizes, for example, due to the different diameters of the insertion and compressible portions of the sleeve, as well as the sealing mechanism of the sleeve. Moreover, by externally sealing the incision to prevent fluid leakage at the incision, and also the larger diameter of the compressible portion of the sleeve (which provides a greater infusion fluid volume nearer the eye, compared to a conventional sleeve), fluidics stability may be improved. Further, a relatively smaller length of the sleeve of the present disclosure enters the eye which may provide stronger structural strength to facilitate ease of insertion. And, to use the infusion sleeve of the present disclosure, no change in existing surgical technique is needed.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Specific dimensions, specific materials, and/or specific shapes disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” as well as the phrase “at least one of” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper”, “lower” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the present disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the present disclosure, and all such modifications are intended to be included within the scope of the present disclosure.

What is claimed is:

1. An ophthalmic surgical instrument comprising:
a needle for insertion into an incision in an eye;
a generator of ultrasonic energy attached to the needle for supplying the ultrasonic energy to the needle to emulsify tissue within the eye; and
a sleeve surrounding at least a portion of the needle, the sleeve including a distal insertion portion for supplying infusion fluid into the eye and a compressible portion for sealing the incision in the eye by pushing against an exterior surface of the eye at the incision.

2. The ophthalmic surgical instrument of claim 1, wherein a diameter of the insertion portion of the sleeve is smaller than a diameter of the compressible portion of the sleeve.

3. The ophthalmic surgical instrument of claim 1, wherein the compressible portion of the sleeve includes a flange at a distal end of the compressible portion to enhance the seal at the incision.

4. The ophthalmic surgical instrument of claim 1, wherein the first diameter of the sleeve's insertion portion is sized to be inserted through the incision and does not seal the incision of the eye.

5. The ophthalmic surgical instrument of claim 1, wherein the insertion portion of the sleeve includes at least one infusion port for supplying the infusion fluid into the eye.

6. The ophthalmic surgical instrument of claim 1, wherein the second diameter of the sleeve's compressible portion is sized to be large enough not to pass through the incision and the compressible portion is configured to remain external to the eye during surgery.

7. The ophthalmic surgical instrument of claim 1, wherein the sleeve is configured to allow the needle to move within the eye independent of the sleeve.

8. An infusion sleeve for a phacoemulsification handpiece, the infusion sleeve comprising:
an insertion portion, wherein the insertion portion has at least a first diameter sized to be inserted through an incision of the eye and does not seal the incision of the eye; and
a compressible portion for sealing against an exterior surface of the eye at the incision, wherein the compressible portion has a second diameter that is larger than the first diameter.

9. The infusion sleeve of claim 8, wherein the insertion portion includes at least one infusion port for supplying infusion fluid into the eye.

10. The infusion sleeve of claim 8, wherein the compressible portion includes a flange at a distal end of the compressible portion to seal the incision by pressing against the exterior surface of the eye.

11. The infusion sleeve of claim 8, wherein the second diameter of the sleeve's compressible portion is sized to be large enough not to pass through the incision and the compressible portion is configured to remain external to the eye during surgery.
